(19) United States
(12) Patent Application Publication
(10) Pub. No.: US 2026/0207720 A1
(43) Pub. Date: Jul. 23, 2026

(54) COMPOSITIONS AND METHODS FOR TREATING FUNGAL INFECTIONS

(71) Applicant: MUCPharm Pty Ltd, Malvern, Victoria (AU)

(72) Inventor: David Morris, Malvern, Victoria (AU)

(21) Appl. No.: 19/134,198

(22) PCT Filed: Dec. 4, 2023
(86) PCT No.: PCT/AU2023/051249

§ 371 Date: May 29, 2025

(30) Foreign Application Priority Data
Dec. 2, 2022 (AU) 2022903688

(51) Int. Cl. A61K 38/48; A61K 31/198; A61K 31/4196; A61K 31/4439; A61K 31/496; A61K 31/7048; A61P 31/10

(52) U.S. Cl. CPC A61K 38/4873; A61K 31/198; A61K 31/4196; A61K 31/4439; A61K 31/496; A61K 31/7048; A61P 31/10

(57) ABSTRACT

Disclosed herein is a method for treating a fungal infection in a patient, the method comprising administering to the patient a therapeutically effective combination of a glycoprotein affecting protease and an antifungal agent. Also disclosed is a method for sensitizing a fungus having a glycoprotein-containing fungal cell wall to an antifungal agent. The method comprises contacting the fungus with a glycoprotein affecting protease.

### CROSS REFERENCE TO RELATED APPLICATION

This PCT application claims priority from Australian provisional patent application no. 2022903688, the contents of which are incorporated herein in their entirety.

### TECHNICAL FIELD

The present invention relates to compositions and methods for treating fungal infections. The present invention also relates to sensitising certain fungi to antifungal agents.

### BACKGROUND ART

Fungal infections are common and can vary in severity from being a mere nuisance to being an immediately life threatening disease. Indeed, fungal infections are reported to kill more than 1.5 million people per year and affect over a billion people.

Fungal infections occur when fungal spores enter a patient's body, generally via the respiratory tract (i.e. via inhalation) or via a wound. Fungal infections include candidiasis (caused by Candida), cryptococcosis (caused by Cryptococcosis neoformans), histoplasmosis (caused by Histoplasma), pneumocystis (caused by Pneumocystis jirovecii) and mucormycosis (caused by mucormycetes).

Many antifungal agents are prescribed to treat a variety of fungal infections. However, the toxicity of such agents can often limit their tolerated dosage (particularly for critically ill patients), reducing their effectiveness. Furthermore, antifungal resistance is becoming ever more common, and is a particularly serious problem with some invasive fungi. Some fungi are also naturally resistant to antifungal agents (e.g. fluconazole is not active in Aspergillus), whilst an acquired resistance can develop over time in other fungi, especially in Aspergillus and Candida.

It would be advantageous to provide new therapeutic options for treating fungal infections.

### SUMMARY OF INVENTION

In a first aspect, the present invention provides a method for treating a fungal infection in a patient. The method comprises administering to the patient a therapeutically effective combination of a glycoprotein affecting protease and an antifungal agent.

In a second aspect, the present invention provides a method for sensitizing a fungus which is the cause of a fungal infection in a patient to an antifungal agent, wherein the fungus has a glycoprotein-containing fungal cell wall. The method comprises administering to the patient a glycoprotein affecting protease.

In a third aspect, the present invention provides a method for sensitizing a fungus having a glycoprotein-containing fungal cell wall to an antifungal agent. The method comprises contacting the fungus with a glycoprotein affecting protease.

In a fourth aspect, the present invention provides a method for treating a fungal infection in a patient. The method comprises administering to the patient a therapeutically effective combination of a glycoprotein affecting protease and a disulphide bond breaking agent.

In a fifth aspect, the present invention provides the use of a glycoprotein affecting protease to sensitize a fungus having a glycoprotein-containing fungal cell wall, whereby efficacy of an antifungal agent is increased.

In a sixth aspect, the present invention provides a composition comprising a glycoprotein affecting protease and an antifungal agent.

In a seventh aspect, the present invention provides a synergistic combination of a glycoprotein affecting protease and an antifungal agent.

In an eighth aspect, the present invention provides a synergistic combination of a glycoprotein affecting protease and an antifungal agent, when used to sensitize a fungus having a glycoprotein-containing fungal cell wall to an antifungal agent.

In a ninth aspect, the present invention provides the use of a combination of a glycoprotein affecting protease and an antifungal agent for the preparation of a medicament for the treatment of a fungal infection in a patient.

In a tenth aspect, the present invention provides a combination of a glycoprotein affecting protease and an antifungal agent for use in medicine.

In an eleventh aspect, the present invention provides a combination of a glycoprotein affecting protease and an antifungal agent for use in the treatment of a fungal infection in a patient.

The use of bromelain (a particular glycoprotein affecting protease) as an antifungal agent has been reported in the context of limiting the growth of certain plant fungi. However, this effect is reportedly not widespread across many varieties of fungi. Indeed, experiments performed by the inventors, some of which are described below, showed that bromelain alone appears to have no appreciable antifungal activity on Rhizopus microspores, Fusarium solani cx, Rhizopus arrhizus and Candida krusei QC. To the contrary, the use of bromelain in some of these fungi was found to stimulate fungal growth under the experimental conditions used.

It was therefore surprising to the inventors that combinations of bromelain and a number of kinds of antifungal agents were found to have a synergistic effect on the fungi that is responsible for mucormycosis. The inventors are not aware of any suggestion in the literature that bromelain might improve the effect of antifungal agents, or otherwise sensitise fungi, specifically fungi that are capable of infecting humans.

Experiments conducted by the inventors following their initial discovery have shown that the co-administration of bromelain and various antifungal agents results in an increased sensitivity of the antifungal agent (in some cases, a clear synergy was observed) in a wide variety of Mucor isolates and in different types of Candida. As would be appreciated, Candida is a yeast and Mucor is a mold, so the invention appears to have applicability to multiple types of fungus. The inventors observed differing levels of sensitivity with different types of fungus, and speculate (without wishing to be bound by theory) that the fungal cell wall may be responsible for this difference. As a person skilled in the art would appreciate, the fungal cell wall is a complex cellular organelle composed of glucans, chitin, chitosan, and other glycosylated proteins. The proportions of these components vary between different types of fungi, which the inventors believe may be the reason for the varying sensitivities they observed in their preliminary work.

Generally speaking, and based on only their preliminary data, the inventors speculate that the changes in efficacy they have observed in performing the various embodiments of the present invention with different fungi (some of which are described below), depend on the proportion of glycoprotein in the cell walls of the specific fungus, as well as the accessibility of those glycoproteins.

As would be appreciated by a person skilled in the art, cell wall proteins have different functions, including the maintenance of the cellular shape, adhesion processes, cellular protection against different substances, absorption of molecules, signal transmission and synthesis and reorganization of wall components. Glycoproteins reportedly compose 30-50% of the dry weight of fungal wall in yeast and 20-30% of the dry weight of the wall of filamentous fungi. The chitin content of fungal cell walls reportedly varies according to the morphological phase of the fungus, representing 1-2% of the dry weight of yeast cell wall and up to 10-20%. in filamentous fungi. Other fungi, such as lomentaspora and scedosporium, for example, have cell walls with a protective layer of melanin.

Again, without wishing to be bound by theory, the inventors believe that the improved sensitivity of fungus to antifungal agents that they have observed arises because bromelain affects the glycoproteins (at least) and glycans in fungal cell walls containing such, dissolving or otherwise degrading the cell walls such that the antifungal agent has greater access to the cell, hence its enhanced therapeutic effect. The inventors predict that the mechanism of action of bromelain in the sensitisation of fungus is likely to be related to dissolution of glycoproteins in the fungal wall (although this has never been previously studied to their knowledge), and hence the likely general applicability of this invention (i.e. utilising any glycoprotein affecting protease) to any fungus having a glycoprotein-containing fungal cell wall.

As noted above, invasive fungal diseases have high mortalities and limited therapies. Treatment in accordance with the present invention has the potential for an increased efficacy, or at least a maintained efficacy using a reduced dose of antifungal agent, thereby limiting their side effects.

The inventors believe that the results of their preliminary experiments (some of which are described below) lead to a reasonable prediction that glycoprotein affecting proteases in addition to bromelain, including those described herein, may also be effective for therapeutic applications such as those described herein. Further experiments, some of which are currently underway, should confirm the inventors' predictions.

The encouraging results from the inventors' early experiments prompted them to revisit the antifungal activity of combinations of glycoprotein affecting proteases and disulphide bond breaking agents, specifically in the form of a combination of bromelain and N-acetylcysteine, a known therapeutic branded as BromAc®. The inventors were surprised to find that this combination did exhibit an antifungal effect, particularly in fungi having cell walls that includes relatively high proportions of glycoproteins and polysaccharides. Again, without wishing to be bound by theory, the inventors believe that contact between the fungal cell wall and BromAc results in the degradation of glycoproteins and other components of the cell wall. Such degradation weakens the wall, either to an extent where the fungus is no longer viable or to an extent sufficient to allow entry of the bromelain and/or N-acetylcysteine (and, optionally, an antifungal agent) into the cell, killing it.

In some embodiments, the fungal infection may be caused by a fungus from a fungal genus selected from one or more of: Rhizopus, Mucor, Rhizomucor, Cryptococcus, Candida, Syncephalastrum, Cunninghamella, Apophysomyces, Aspergillus, Histoplasma, Pneumocystis and Lichtheimia.

In some embodiments, the fungal infection may be caused by a fungus selected from one or more of: Rhizopus arrhizus, Rhizopus microsporus, Aspergillus fumigatis, Aspergillus flavus, Aspergillus niger, Fusarium solani, Candida krusei, Candida auris, Candida parapslosis, Candida tropicalis, Candida glabrata, Candida albicans, Cryptococcus gattii, Cryptococcus neoformans, Cunninghamella bertholletiae, Lichtheimia corymbifera, Mucor circinelloides, Histoplasma capsulatum, Pneumocystis jirovecii and Microsporidia.

In some embodiments, the fungal infection may, for example, be selected from one or more of: mucormycosis (e.g. cutaneous mucormycosis, Rhino-cerebral mucormycosis and pulmonary mucormycosis), histoplasmosis, cryptococcosis, pneumocystis pneumonia, candidiasis and aspergillosis. More generally speaking, the present invention might be used to treat systemic fungal infections, such as systemic fungal sepsis, as well as localised fungal infections, such as the infections found on prosthesis or in the respiratory tract, for example. Any respiratory infection from fungi might also be treated using the present invention, noting that Aspergillus, Candida and Mucorales are particularly lung invasive.

In some embodiments, the glycoprotein affecting protease and the antifungal agent may be administered to the patient simultaneously or sequentially. The glycoprotein affecting protease and the antifungal agent may, for example, be co-administered to the patient. The glycoprotein affecting protease and the antifungal agent may, for example, be administered to the patient via different routes of administration. Potential routes of administration include locally, systemically, intravenously, by inhalation, by nebulization, by intratracheal injection, by intracavitary injection, by intraperitoneal injection, by intrapleural injection, by intraocular injection, by injection into the parenchyma or by injection into the cerebrospinal fluid via the intracerebroventricular or intrathecal (cisternal or lumbar) route, for example.

In some embodiments, the glycoprotein affecting protease may be a cysteine protease. In some embodiments, the glycoprotein affecting protease may be selected from one or more of: bromelain, papain, ficain, actinidain, zingibain, fastuosain and ananain.

In some embodiments, the antifungal agent may be selected from one or more of the group consisting of: Polyenes (e.g. amphotericin B and nystatin), Echinocandins (e.g. caspofungin and micofungin), Azoles (e.g. isavuconazole, posaconazole, fluconazole, ketaconazole, voriconazole and itraconazole), Allylamines (e.g. Terbinafine) and Ortomides. Emerging antifungal agents, including inhibitors of calcineurin, trehalose pathway inhibitors and inhibitors of sphingolipid synthesis, might also potentially be used in the present invention.

In some embodiments, the invention may further comprise administering to the patient a disulphide bond breaking agent (which may also function as a thiol donor) in combination with the glycoprotein affecting protease and antifungal agent (or just the glycoprotein affecting protease in embodiments of the fourth aspect of the present invention). Synergy between glycoprotein affecting proteases such as bromelain and disulphide bond breaking agents such as N-acetylcysteine and cysteamine has, for example, been observed previously by some of the present inventors.

In such embodiments, the disulphide bond breaking agent may, for example, be selected from one or more of: N-acetylcysteine, cysteamine, erdosteine, s-carboxymethylcysteine, glutathione, dithiothreitol, nacystelyn, mercapto-ethanesulphonate, carbocysteine, dornase alfa, gelsolin, thymosin P4, dextran, bucillamine, dithiobutylamine (DTBA) and heparin. In such embodiments, the glycoprotein affecting protease and disulphide bond breaking agent may be administered simultaneously or sequentially.

In some embodiments, the invention may further comprise administering to the patient an additional agent (e.g. DNase or Collagenase). Such an additional agent(s) may help to even further degrade the fungal cell components, thus even further enhancing the antifungal agent's effect.

In some embodiments, the invention may further comprise administering to the patient one or more additional therapeutic agents. In such embodiments, the one or more additional therapeutic agents may be selected from the group consisting of antiviral agents, antibacterial agents, bronchodilators and expectorants.

It is to be understood that any features and embodiments described herein in detail in relation to a specific aspect of the invention are equally applicable to other aspects of the invention. Other aspects, features and advantages of the present invention will be described below.

### BRIEF DESCRIPTION OF THE DRAWINGS

Embodiments of the present invention will now be described in further detail with reference to the following drawings, in which the p-values were used to measure significant killing over the growth control and, in the graphs: *p<0.05, **p<0.01, ***p<0.001, and ****p<0.0001. All doses presented in are in μg/ml, except for Acetylcysteine which is in mg/ml.

FIG. 1 shows (A) a dose response graph which demonstrates the percent growth of C. glabrata when exposed to Bromelain alone, and to Bromelain in combination with Amphotericin B; and (B) shows a dose response graph which demonstrates the percent growth of C. glabrata when exposed to Amphotericin B alone, (GC=Growth Control, B=Bromelain and A=Amphotericin B).

FIG. 2 shows a dose response graph which demonstrates the percent growth of C. glabrata when exposed to Amphotericin B alone (GC=Growth Control; A=Amphotericin B).

FIG. 3 shows a dose response graph which demonstrates the percent growth of C. glabrata when exposed to Amphotericin B, BromAc and BromAc with Amphotericin B (GC=Growth Control, A=Amphotericin B, B=Bromelain and N=Acetylcysteine). The a) and b) represent the higher and lower concentrations of Bromelain used.

FIG. 4 shows a dose response graph which demonstrates the percent growth of C. glabrata when exposed to Posaconazole alone (GC=Growth Control and P=Posaconazole).

FIG. 5 shows a dose response graph which demonstrates the percent growth of C. glabrata when exposed to Posaconazole, BromAc and BromAc with Posaconazole (GC=Growth Control, P=Posaconazole, B=Bromelain and N=Acetylcysteine). The a) and b) represent the higher and lower concentrations of Bromelain used.

FIG. 6 shows a dose response graph which demonstrates the percent growth of C. glabrata when exposed to fluconazole alone (GC=Growth Control and P=Posaconazole).

FIG. 7 shows a dose response graph which demonstrates the percent growth of C. glabrata when exposed to Fluconazole, BromAc and BromAc with Fluconazole (GC=Growth Control, F=Fluconazole, B=Bromelain and N=Acetylcysteine). The a) and b) represent the higher and lower concentrations of Bromelain used.

FIG. 8 shows a dose response graph which demonstrates the percent growth of C. Krusei when exposed to Amphotericin B alone (GC=Growth Control and A=Amphotericin B).

FIG. 9 shows a dose response graph which demonstrates the percent growth of C. Krusei when exposed to Amphotericin B, BromAc and BromAc with Amphotericin B (GC=Growth Control, A=Amphotericin B, B=Bromelain and N=Acetylcysteine). The a) and b) represent the higher and lower concentrations of Bromelain used.

FIG. 10 shows (a) a dose response graph which demonstrates the percent growth of C. Krusei when exposed to Posaconazole alone at various doses and (b) shows a dose response graph which demonstrates the percent growth of C. Krusei when exposed to Posaconazole, BromAc and BromAc with Posaconazole (GC=Growth Control, P=Posaconazole, B=Bromelain and N=Acetylcysteine).

FIG. 11 shows a dose response graph which demonstrates the percent growth of C. Krusei when exposed to Fluconazole alone (GC=Growth Control and F=Fluconazole).

FIG. 12 shows a dose response graph which demonstrates the percent growth of C. Krusei when exposed to Fluconazole, BromAc and BromAc with Amphotericin B (GC=Growth Control, F=Fluconazole, B=Bromelain and N=Acetylcysteine). The a) and b) represent the higher and lower concentrations of Bromelain used.

FIG. 13 shows (a) a dose response graph which demonstrates the percent growth of Aspergillus fumigatus when exposed to Voriconazole alone at various doses and (b) shows a dose response graph which demonstrates the percent growth of Aspergillus fumigatus when exposed to Voriconazole, BromAc and BromAc with Voriconazole (GC=Growth Control, V=Voriconazole, B=Bromelain and N=Acetylcysteine).

FIG. 14 shows (a) a dose response graph which demonstrates the percent growth of Aspergillus fumigatus when exposed to Caspofungin alone at various doses and (b) shows a dose response graph which demonstrates the percent growth of Aspergillus fumigatus when exposed to Caspofungin, BromAc and BromAc with Caspofungin (GC=Growth Control, C=Caspofungin, B=Bromelain and N=Acetylcysteine).

### DETAILED DESCRIPTION OF THE INVENTION

As noted above, in its most general form, the present invention provides methods for treating fungal infections in a patient, in which methods the patient is administered a therapeutically effective combination of a glycoprotein affecting protease and an antifungal agent. The present invention also provides methods for sensitizing fungus having a glycoprotein-containing fungal cell wall to an antifungal agent, in which methods the fungus is contacted with a glycoprotein affecting protease. The present invention also provides methods for sensitizing a fungus which is the cause of a fungal disease or infection in a patient to an antifungal agent, wherein the fungus has a glycoprotein-containing fungal cell wall, in which method the patient is administered a glycoprotein affecting protease. Finally, the present invention also provides method for treating a fungal infection in a patient, in which methods the patient is administered a therapeutically effective combination of a glycoprotein affecting protease and a disulphide bond breaking agent.

Fungal infections expected to be treatable in accordance with the present invention include mucormycosis (e.g. cutaneous mucormycosis, Rhino-cerebral mucormycosis and pulmonary mucormycosis), histoplasmosis, cryptococcosis, pneumocystis pneumonia, candidiasis and aspergillosis. The present invention could also potentially be used to treat systemic fungal infections, such as systemic fungal sepsis, as well as localised fungal infections, such as the infections found on prosthesis. Any respiratory infection from fungi might also be treated using the present invention, noting that Aspergillus, Candida and Cryptococcus are particularly lung invasive.

Such fungal infections may be caused by fungus from fungal genus including: Rhizopus, Mucor, Rhizomucor, Cryptococcus, Candida, Syncephalastrum, Cunninghamella, Apophysomyces, Aspergillus, Histoplasma, Pneumocystis and Lichtheimia. Specific fungus responsible for these fungal infections may include: Rhizopus arrhizus, Rhizopus microsporus, Aspergillus fumigatis, Aspergillus flavus, Aspergillus niger, Fusarium solani, Candida krusei, Candida auris, Candida parapslosis, Candida tropicalis, Candida glabrata, Candida albicans, Cryptococcus gattii, Cryptococcus neoformans, Cunninghamella bertholletiae, Lichtheimia corymbifera, Mucor circinelloides, Histoplasma capsulatum, Pneumocystis jirovecii and Microsporidia.

In some embodiments, the patient may be a mammalian subject. Typically, the patient will be a human patient, although other subjects may benefit from the present invention. For example, the subject may be a pig, mouse, rat, dog, cat, cow, sheep, horse or any other mammal of social, economic or research importance.

As described above, the inventors predict that the mechanism of action of bromelain in the observed sensitisation of antifungals is likely to be related to dissolution of glycoproteins (and potentially other components such as glucan and chitin) in the fungal wall, although this has not previously been studied to their knowledge. The inventors believe that the results of their preliminary experiments, some of which are described below, support a reasonable prediction of the broad therapeutic applications disclosed herein. The inventors also believe that the results of their preliminary experiments support a reasonable prediction that other glycoprotein affecting proteases will have utility in the present invention. Further experiments along the lines of those described herein should confirm the inventors' predictions.

### Glycoprotein Affecting Proteases

The present invention involves the use of a glycoprotein affecting protease. Glycoprotein affecting proteases are proteolytic enzymes which cause proteolysis of glycoproteins. Given their preliminary data for bromelain, which is a protease enzyme that affects glycoproteins by hydrolysing peptide and glycosidic bonds within the glycoproteins, the inventors believe that any glycoprotein affecting protease may be used in the present invention, with routine trial and experimentation being all that would be required (in light of the teachings contained herein) in order to determine any particular glycoprotein affecting protease's suitability. As used herein, the term “Glycoprotein affecting” is to be understood as affecting the glycoproteins (such as chitin, for example) in the fungal cell wall in any therapeutically effective manner such as, for example, by digesting, liquefying or otherwise causing the glycoprotein (as well as other peptide bonds) in the cell wall to disintegrate or degrade. The glycoprotein affecting protease may, for example, be effective to disintegrate glycoproteins. The glycoprotein affecting protease may, for example, be effective to hydrolyse peptide and glycosidic bonds of glycoproteins.

As noted above, glucans, particularly β-glucans are another major component in fungal cell walls. An enhanced therapeutic effect may therefore be achieved if a glucan affecting agent is also included in the present invention. In some embodiments, the glycoprotein affecting protease may also be a glucan affecting protease (e.g. as is the case for bromelain). In alternative embodiments, glucan affecting agents, such as poacic acid and echinocandins for example, may be used in combination with the glycoprotein affecting protease.

The glycoprotein affecting protease may, for example, be a cysteine protease. Cysteine proteases (also known as thiol proteases) degrade proteins via a common catalytic mechanism, and are commonly sourced from fruits including the papaya, pineapple, fig and kiwifruit. Examples of cysteine proteases include bromelain, papain (extracted from papaya) and ananain, a plant cysteine protease in the papain superfamily of cysteine proteases.

There are other plant-derived proteolytic enzymes that express the same characteristics as bromelain and the inventors expect that any plant-derived protease enzymes, or recombinants, which have an effect on glycoproteins may be used in the present invention. Again, routine experimentation should be able to confirm the suitability of any particular plant-derived protease enzyme. In some embodiments, for example, the plant-derived protease enzymes may be selected from one or more of the group consisting of Bromelain, Papain (extracted from papaya), Ficain (extracted from figs), Actinidain (extracted from fruits including kiwifruit, pineapple, mango, banana and papaya), Zingibain (extracted from ginger) and Fastuosain (a cysteine proteinase from Bromelia fastuosa). Asparagus, mango and other kiwi fruit and papaya proteases may also be useful.

Active fractions of glycoprotein affecting proteases may be used in the present invention, noting that it may not be necessary for all substances in the protease extract to be included, provided that the fraction itself affects the glycoprotein. It is expected that glycoprotein affecting protease enzymes obtained using genetic recombination may also be used in the present invention.

As used herein, “Bromelain” is to be understood to encompass one or more of the glycoprotein affecting and, optionally, otherwise therapeutically active substances present in the extract of the pineapple plant (Ananas Comosus). Bromelain is a mixture of substances (including different thiol endopeptidases and other components such as phosphatase, glucosidase, peroxidase, cellulase, esterase and several protease inhibitors) and it may not be necessary for all of these substances to be included in the combination, provided that the fraction of the substances in the combination can at least affect the glycoproteins.

The Bromelain used in the experiments described herein was commercially sourced from Enzybel Group, with any further extraction and purification processing being performed by Mucpharm Pty Ltd.

The amounts and relative proportions of the glycoprotein affecting protease and antifungal agent in the present invention may vary depending on factors such as the kinds of glycoprotein affecting protease and antifungal agent, their intended use, as well as patient factors such as their weight and other health factors including the severity of infection. Generally speaking, however, the combinations or compositions for administration would be expected to include between about 5 μg/mL and about 2 mg/mL of the glycoprotein affecting protease. These amounts are higher than may be needed to achieve a therapeutic effect for some routes of administration, but the inventors note that only a fraction of drug administered by some routes (e.g. nebulisation) may become bioavailable. As losses of drug may vary between treatment regimens and apparatus, the amounts of glycoprotein affecting protease described below refer to that which will be received by the patient, such being measurable using routine techniques.

Amounts of glycoprotein affecting protease lower than about 5 μg/mL may not be effective and amounts higher than about 1,000 μg/mL would be more likely to cause undesirable side effects systemically. In some embodiments for example, about 5 μg/mL, 10 μg/mL, 15 μg/mL, 20 μg/mL, 30 μg/mL, 40 μg/mL, 50 μg/mL, 60 μg/mL, 80 μg/mL, 100 μg/mL, 150 μg/mL, 200 μg/mL, 250 μg/mL, 300 μg/mL, 350 μg/mL, 400 μg/mL, 450 μg/mL, 500 μg/mL, 550 μg/mL, 600 μg/mL, 650 μg/mL, 700 μg/mL, 750 μg/mL, 800 μg/mL, 850 μg/mL, 900 μg/mL, 950 μg/mL, 1,000 μg/mL of the glycoprotein affecting protease may be administered in the combination.

It is envisaged that, for some therapies and/or patients, repeated treatments may be beneficial (or necessary) in order to complete an effective treatment.

The glycoprotein affecting protease and antifungal agent may be administered to the patient in any manner that provides the intended therapeutic effect. They may, for example, be simultaneously administered (e.g. in a single composition), sequentially administered (e.g. in separate compositions, one after the other) or separately administered (e.g. in separate compositions), potentially via different routes of administration.

### Antifungal Agent

Any antifungal agent is likely to be able to be used in the present invention, with routine trial and experimentation being all that would be required to determine any given antifungal agent's suitability. Examples of potentially suitable antifungal agents include Polyenes (e.g. amphotericin B, liposomal amphotericin and nystatin), Echinocandins (e.g. caspofungin and micofungin), Azoles (e.g. posaconazole, isavuconazole, fluconazole, ketaconazole, voriconazole and itraconazole), Allylamines (e.g. Terbinafine) and Ortomides. As would be appreciated, some fungal infections are treated with multiple classes of antifungal agents (e.g. a polyene plus an azole), and such may also be the case in the present invention.

Emerging antifungal agents including inhibitors of calcineurin, trehalose pathway inhibitors and inhibitors of sphingolipid synthesis might also potentially be used in the present invention. Other antifungal agents, such as fungal cytochrome P450 enzyme inhibitors, B-glucan synthesis inhibitors, chitin synthase inhibitors, Ergosterol binding agents, squalene epoxidase inhibitors, alcineurin signalling inhibitors, DNA synthesis inhibitors, Hsp90 inhibitors, protein synthesis inhibitors, microtubule assembly acting, ROS (reactive oxygen species) producer might potentially give synergistic effect in treatment.

In use, the antifungal agent achieves its indicated therapeutic effect (at least for the experiments conducted by the inventors, see below), but synergy has been observed with significantly reduced amounts of the antifungal agent being required for the same therapeutic outcome. As described above, given that many antifungal agents have undesirable side effects, lower dosages may be highly advantageous.

As noted above, the amounts and relative proportions of the glycoprotein affecting protease and antifungal agent in the present invention may vary depending on a number of factors. Generally speaking, however, the combinations or compositions for administration would be expected to include amounts of the antifungal agents that are significantly lower than that needed for the antifungal agent to achieve its therapeutic effect if administered alone (i.e. conventionally). It is within the ability of a person skilled in the art to ascertain an appropriate amount of the antifungal agent based on the teachings contained herein and the information available from standard medical sources. For example, amphotericin is indicated for treatment of Aspergillus, Candida, and/or Cryptococcus species, at a dose rate of 3-5 mg/kg IV qDay.

### Disulphide Bond Breaking Agent

In some embodiments, the invention may further comprise administering a disulphide bond breaking agent to the patient in combination with the glycoprotein affecting protease and antifungal agent (or with the glycoprotein affecting protease only, in accordance with such aspects of the present invention). Disulphide bond breaking agents are species that break the disulphide bridges that help to define the tertiary structure of proteins.

In such embodiments, the disulphide bond breaking agent may, for example, be selected from one or more of: N-acetylcysteine, cysteamine, erdosteine, s-carboxymethylcysteine, glutathione, dithiothreitol, nacystelyn, mercapto-ethanesulphonate, carbocysteine, N-acystelyn, dornase alfa, gelsolin, thymosin P4, dextran, bucillamine, dithiobutylamine (DTBA) and heparin. In such embodiments, the glycoprotein affecting protease and disulphide bond breaking agent may be administered simultaneously or sequentially.

In some embodiments for example, about 2.5 mg/ml (0.25% w/v), 5 mg/ml (0.5% w/v), 10 mg/ml (1.0% w/v), 15 mg/ml (1.5% w/v) or 20 mg/ml (2.0% w/v), of a disulphide bond breaking agent such as N-acetylcysteine may be included in the combination or composition for administration.

The term “BromAc”, as used herein, is a combination of bromelain and acetylcysteine, which is a drug combination that has been developed by some of the present inventors for treating mucinous cancers. BromAc was found to rapidly dissolve and remove tumour mucin, whilst neither of the drugs worked alone.

Additional Agents that Degrade Fungal Cell Walls

In some embodiments, the invention may further comprise administering to the patient an additional agent or agents (i.e. in combination with the glycoprotein affecting protease and antifungal agent (or with the glycoprotein affecting protease and disulphide bond breaking agent, in such aspects of the present invention)) that may contribute to degrading fungal cell walls (e.g. Dnase or collagenase). Such an additional agent(s) may help to even further degrade (or more quickly degrade) the fungal cell wall (e.g. Dnase may degrade any DNA in the fungal cell), thus even further enhancing the antifungal's effect.

In compositions including an additional fungal cell wall degrading agent, the agent may be present in the composition in any amount that produces a beneficial effect. For example, in the case of Dnase, an amount of from about 5 to 200 μg/mL would be expected to provide the beneficial effects described herein.

### Additional Therapeutic Agents

The present invention may also include an additional therapeutic agent. Any additional therapeutic agent having an appropriate indication in the context of treating fungal infections may also be co-administered to the patient. Examples of additional therapeutic agents include antiviral agents, antibacterial agents, bronchodilators and/or expectorants. The additional therapeutic agent may be simultaneously or (more likely) sequentially administered with the glycoprotein affecting protease and/or antifungal agent.

In compositions including an additional therapeutic agent, any amount of the agent that produces a beneficial effect may be used. It is within the ability of a person skilled in the art to determine an appropriate quantity of any such additional therapeutic agent. In some embodiments, two or more additional therapeutic agents may provide beneficial effects, especially if their therapeutic effect is via different mechanisms.

Other components may be included in the present invention which do not necessarily have a direct therapeutic effect. Such components include pharmaceutically acceptable excipients and carriers.

### Administration

The glycoprotein affecting protease and antifungal agent may be administered to a patient in any manner that provides the intended therapeutic effect. Any administration route that results in the glycoprotein affecting protease (and other components of the combination) coming into proximity to the fungus is expected to be effective. Potential routes of administration include locally, systemically, intravenously, by inhalation, by nebulization, by intratracheal injection, by lung lavage, by intracavitary injection, by intraperitoneal injection, by intrapleural injection, by intraocular injection, by injection into the parenchyma or by injection into the cerebrospinal fluid via the intracerebroventricular or intrathecal (cisternal or lumbar) route, for example.

In some embodiments, the glycoprotein affecting protease and the antifungal agent may be administered simultaneously or sequentially. The glycoprotein affecting protease and the antifungal agent may, for example, be co-administered to the patient. The glycoprotein affecting protease and the antifungal agent may, for example, be administered to the patient via different routes of administration.

If administered into the lungs of the patient (e.g. after being nebulized), for example to treat mucormycosis, the combination may, for example, be sprayed into the trachea or bronchi using specialised medical equipment, such as via a bronchoscope. Alternatively (or in addition), the combination may be sprayed into the patient's nose or mouth or trachea when they are inhaling via nebulisation. Alternatively (or in addition), the combination may be nebulised and delivered into an atmosphere surrounding a patient such as a closed system tent or other closed-in environmental spaces for treatment.

Nebulisation is a commonly used method for delivering drugs into the respiratory tract. Nebulisers are delivery devices used to administer medication in the form of a mist inhaled into the lungs, and can use oxygen, compressed air or ultrasonic power to break up solutions and suspensions into small aerosol droplets that are inhaled from the mouthpiece of the device.

Another commonly used method for delivering drugs into the respiratory tract is to use a dry powder inhaler or metered dose inhaler. Such inhalers are well known in the art and deliver a specific amount of medication to the lungs, in the form of a short burst of aerosolized medicine that is usually self-administered by the patient via inhalation.

The dosage of the combination of the present invention administered to a patent will depend on factors such as the route of administration, severity of infection and weight of the patient. It would be within the ability of a person skilled in the art to determine an appropriate dosage (and dosage regimen) based on the teachings contained herein and their experience.

### Pharmaceutical Compositions

The combination of glycoprotein affecting protease, antifungal agent and optional further agents used in the present invention may, in some embodiments, be provided in the form of a pharmaceutical composition comprising a pharmaceutically acceptable carrier.

Such a pharmaceutically acceptable carrier will depend on the route of administration of the composition. Liquid form preparations may include solutions, suspensions and emulsions, for example water or water-propylene glycol solutions for creating aerosols for airway (intranasal or intratracheal) delivery. Suitable pharmaceutically acceptable carriers for use in the pharmaceutical compositions of the present invention include physiologically buffered saline, normal saline, hypertonic saline, dextrose solutions and Ringer's solution, etc. As noted above, powder formulations for inhalation are also envisaged.

Pharmaceutical compositions suitable for delivery to a patient may be prepared immediately before delivery into the patient's body or may be prepared in advance and stored appropriately beforehand.

Pharmaceutical compositions and medicaments for use in the present invention may comprise a pharmaceutically acceptable carrier, adjuvant, excipient and/or diluent. The carriers, diluents, excipients and adjuvants must be “acceptable” in terms of being compatible with the other ingredients of the composition or medicament and the delivery method, and be generally not deleterious to the recipient thereof.

It will be understood that, where appropriate, some of the components in the combinations or pharmaceutical compositions described herein may be provided in the form of a metabolite, pharmaceutically acceptable salt, solvate or prodrug thereof. “Metabolites” of the components of the invention refer to the intermediates and products of metabolism.

“Pharmaceutically acceptable”, such as pharmaceutically acceptable carrier, excipient, etc., means pharmacologically acceptable and substantially non-toxic to the subject to which the particular compound is administered.

“Pharmaceutically acceptable salt” refers to conventional acid-addition salts or base addition salts that retain the biological effectiveness and properties of the components and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluene sulfonic acid, salicylic acid, methanesulfonic acid, oxalic acid, succinic acid, citric acid, malic acid, lactic acid, fumaric acid, and the like. Sample base-addition salts include those derived from ammonium, potassium, sodium and quaternary ammonium hydroxides, such as for example, tetramethylammonium hydroxide. The chemical modification of a pharmaceutical compound (i.e. drug) into a salt is a technique well known to pharmaceutical chemists to obtain improved physical and chemical stability, hygroscopicity, flow ability and solubility of compounds. See, e.g., H. Ansel et. al., Pharmaceutical Dosage Forms and Drug Delivery Systems (6th Ed. 1995) at pp. 196 and 14561457, which is incorporated herein by reference.

“Prodrugs” and “solvates” of some components are also contemplated. The term “prodrug” means a compound (e.g., a drug precursor) that is transformed in vivo to yield the compound required by the invention, or a metabolite, pharmaceutically acceptable salt or solvate thereof. The transformation may occur by various mechanisms (e.g., by metabolic or chemical processes). A discussion of the use of prodrugs is provided by T. Higuchi and W. Stella, “Prodrugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

### Experimental Results

Experiments conducted by the inventors to demonstrate the effect of specific embodiments of the present invention will now be described.

### Materials

The Bromelain used in the experiments described herein was commercially sourced from Enzybel Group, with any further processing being performed by MUCPharm Pty Ltd, and was provided as a sterile powder. Bromelain was diluted either in phosphate buffered saline (PBS) when used as single agent, or directly in acetylcysteine solution when used in combination (sometimes referred to as “BromAc” in the examples), to prepare formulations of various concentrations. Acetylcysteine (sometimes referred to as “Ac” in the examples) 200 mgmL⁻¹ was purchased from Link Pharma (Australia) and solutions were prepared by dilution in PBS.

Amphotericin B was obtained in aqueous solution (Gibco, Thermo Fisher), while Isavuconazole and Posaconazole were obtained in powder form and dissolved in DMSO at a concentration of 3.2 mgmL⁻¹. The other antifungal drugs described below were obtained in liquid or powder form and dissolved/diluted according to guidelines. All drugs were diluted from DMSO or aqueous stock solutions to appropriate concentrations in RPMI 1640 (Sigma-Aldrich) containing L-glutamine, but lacking sodium bicarbonate, buffered to pH 7.0 with 0.165M MOPS (Sigma-Aldrich).

### Antifungal Susceptibility Testing—Chequerboard Broth Microdilution Method

The in vitro antifungal properties of Bromelain, Acetylcysteine, BromAc, Amphotericin B, Isavuconazole, Fluconazole and Posaconazole were determined using a broth microdilution method in round bottom 96 well plates in accordance with standard procedures defined by the Clinical Laboratory Standards Institute (CLSI). Minimum inhibitory concentrations (MIC) were obtained for each experiment, showing the lowest concentration(s) of drug which resulted in a complete inhibition of growth.

Unless otherwise described, fungal cultures were grown on potato dextrose agar plates at 35° C. until mature (appearance of conidia) and used to prepare suspensions in sterile water. Suspensions were diluted in RPMI 1:60. These suspensions were then added in equal volume to solutions containing the candidate species described above and incubated at 35° C. for 24 hr. Plates were then analysed for fungal growth inhibition and MIC values determined.

The Fractional Inhibitory Concentration (FIC), which indicates the nature of drug interactions between drug A and drug B (with a FIC≤0.5 being indicative of synergy, a FIC >0.5-1 being indicative of an additive effect, a FIC between 1-4 being ‘indifferent’ and a FIC >4 being ‘antagonistic’), may be calculated using the formula:

[MICA (combined)/MICA]+[MICB (combined)/MICB]

MICA (combined) is the MIC of drug A when both drugs (i.e. drug A and drug B) are present and MICA is the MIC of drug A used in isolation. Similarly, MICB (combined) is the MIC of drug B when both drugs are present and MICB is the MIC of drug B used in isolation.

For the bromelain/antifungal agent synergy assays described below, representative strains of various fungi were tested using the chequerboard broth microdilution method with bromelain concentrations of 12.5, 25, 50, 100 and 250 μgmL⁻¹. In experiments that included acetylcysteine, then concentrations of 0.5, 1, 2 and 5 mgmL⁻¹ were used.

### Example 1

Four types of fungi from the Mucormycete group (Rhizopus microspores, Fusarium solani cx, Rhizopus arrhizus and Candida krusei QC (Quality Control), all sourced clinically) were tested with bromelain and acetylcysteine alone. In these experiments, the fungi were observed to all grow well in all concentrations of bromelain tested, with the MIC being greater than 250 μg/mL. Indeed, bromelain in isolation was not observed to display any inhibitory effects at concentrations up to 500 μgmL⁻¹ by susceptibility assays.

The MIC of acetylcysteine in isolation required to inhibit Mucormycete growth was shown by susceptibility assay to be 10 mgmL⁻¹. Initial tests were conducted on one strain of Rhizopus microsporus, one strain of Rhizopus arrhizus and one strain of Candida krusei QC, all sourced clinically.

NAC at 5 mg/mL was used in combination with various concentrations of bromelain (12.5-500 μg/mL) to determine if there was synergy, or at least an additive effect, between these two drugs. Anti-fungal effects on Rhizopus arrhizus and Rhizopus microsporus were observed at 12.5 μg/ml and 25 μg/mL bromelain. These data indicate that bromelain itself has no antifungal effect against the types of fungus described above, whilst NAC has some. The combination of bromelain and NAC, however, appeared to be more effective. These data enabled a MIC to be calculated, which was then tested further with antifungals to examine whether an additional inhibitory effect would be seen.

### Example 2A

Two Rhizopus strains (Rhizopus arrhizus and Rhizopus microspores) were also tested with dilutions of Bromelain (250, 100, 50, 25 μg/mL) in combination with either Amphotericin B (2, 1, 0.5, 0.25, 0.06, 0.03, 0.008, 0.004 mg/L) or Posaconazole (2, 1, 0.5, 0.25, 0.06, 0.03, 0.008, 0.004 mg/L), both of which are indicated as antifungal agents. The results are set out below are very encouraging in that synergy has been demonstrated, the required amount of antifungal is reduced when the Bromelain is added.

From the experimental data, the following calculations can be made:

| Amphotericin B and Bromelain | |
|---|---|
| Rhizopus arrhizus (80-21-015-4777) | |
| Amphotericin: | 0.5 mg/L |
| Bromelain: | >500 mg/L |
| AmB + Br: | 0.03 mg/L of AmB + 25 mg/L of Br |
| | Σ FIC 0.113 (Synergy) |

Rhizopus microsporus (80-21-006-5340)

| | |
|---|---|
| Amphotericin B: | 0.5 mg/L |
| Bromelain: | >500 mg/L |
| AmB + Br: | 0.125 mg/L of AmB + 25 mg/L of Br |
| | Σ FIC 0.3 (Synergy) |

| Posaconazole and Bromelain | |
|---|---|
| Rhizopus arrhizus (80-21-015-4777) | |
| Posaconazole: | 0.5 mg/L |
| Bromelain: | >500 mg/L |
| Posa + Br: | 0.125 mg/L of Posa +50 mg/L of Br |
| | Σ FIC 0.35 (Synergy) |

Rhizopus microsporus (80-21-006-5340)

| | |
|---|---|
| Posaconazole: | 1 mg/L |
| Bromelain: | >500 mg/L |
| Posa + Br: | 0.25 mg/L of Posa + 25 mg/L of Br |
| | Σ FIC 0.3 (Synergy) |

In summary, fungal cultures of R. arrhizus and R. microsporus were grown in multi-well plates and a stepwise reducing concentration of Amphotericin with or without a series of concentration of bromelain from 25-250 μg/mL (equivalent to 25-250 mg/L), showed that bromelain alone at concentrations of 250 and 500 μg/mL had no effect and Amphotericin alone was not effective at 0.25 mg/L. When combined with 25 μg/mL of bromelain, however, complete inhibition was seen at 0.3 mg/L Amphotericin for R. arrhizus and similar results were seen with R. microsporus. Synergy was also seen with Bromelain and Posaconazole in both of the fungal types.

### Example 2B

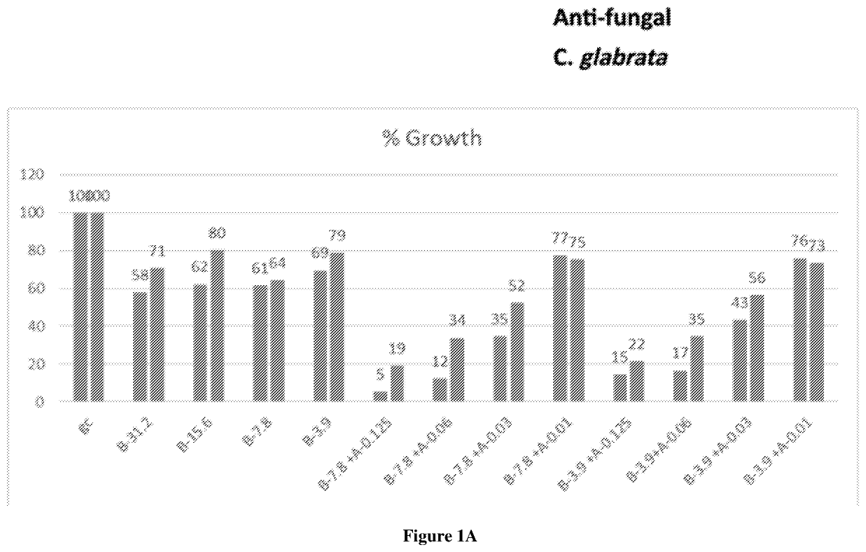

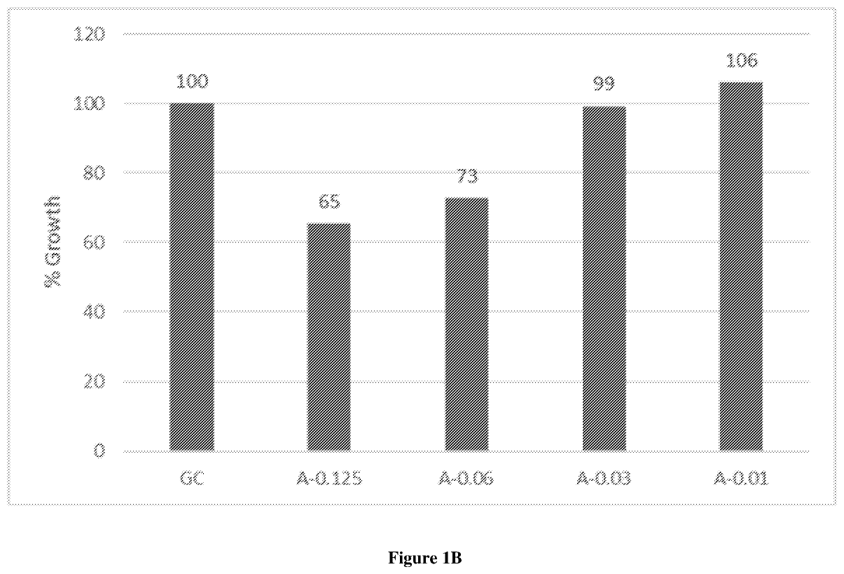

Candida glabrata (ATCC) was also tested with dilutions of Bromelain (31.2, 15.6, 7.8 and 3.9 μg/mL), both in isolation and in combination with Amphotericin B (0.125, 0.06, 0.03 and 0.01 mg/L). The results are set out in FIGS. 1A and 1B and show that MIC is significantly decreased, perhaps even synergistically decreased, when bromelain and Amphotericin B are used in combination.

### Example 3—Bromelain and Acetylcysteine in Combination (“BromAc”) Acts Synergistically with Azole Antifungals to Inhibit Mucoromycete Growth

From data obtained by the inventors, there appeared to be an additive effect between Bromelain and Acetylcysteine at concentrations of 12.5 μgmL⁻¹ and 5 or 10 mgmL⁻¹ respectively. This combination was used for further investigation in combination with Amphotericin B, Isavuconazole, and Posaconazole (three common antifungal agents used in the treatment of mucoromycete infection). The MIC values of these three antifungals in isolation were calculated and compared with the corresponding MICs for the antifungals in the presence of BromAc using standard susceptibility assays.

Eight Rhizopus microsporus and eight Rhizopus arrhizus strains (all sourced from a range of clinical isolates from an Australian hospital) were used in these studies to gain a representative view of the efficacy of these drugs in combination. Each drug combination was tested on all sixteen mucormycete strains and these studies were done in duplicate.

BromAc in Combination with Amphotericin B

For the R. microsporus strains, MICs for Amphotericin B ranged from 0.5-1 μgmL⁻¹ (average 0.7 μgmL⁻¹), and MICs for BromAc ranged from 0.004-0.125 μgmL⁻¹ (average 0.034 μgmL⁻¹) (Table I).

For the R. arrhizus strains, MICs for Amphotericin B ranged from 0.25-1 μgmL⁻¹ (average 0.6 μgmL⁻¹), and MICs with BromAc ranged from 0.008-0.125 μgmL⁻¹ (average 0.05 μgmL⁻¹) (Table I).

Duplicate assays for confirmed this trend.

BromAc in Combination with Isavuconazole

For the R. microsporus strains, MICs for Isavuconazole ranged from 0.5-1 μgmL⁻¹ (average 0.9 μgmL⁻¹), and the MICs with BromAc ranged from 0.03-0.25 μgmL⁻¹ (average 0.13 μgmL⁻¹) (Table I).

For the R. arrhizus strains, MICs for Isavuconazole ranged from 0.5-2 μgmL⁻¹ (average 0.8 μgmL⁻¹), and the MICs with BromAc ranged from 0.06-0.5 μgmL⁻¹ (average 0.17 μgmL⁻¹) (Table I).

Duplicate assays confirmed this trend.

BromAc in combination with Posaconazole

For the R. microsporus strains, MICs for Posaconzole ranged from 0.25-1 μgmL⁻¹ (average 0.5 μgmL⁻¹), and the MICs with BromAc ranged from 0.008-0.08 μgmL⁻¹ (average 0.03 μgmL⁻¹) (Table I).

For the R. arrhizus strains, MICs for Posaconazole ranged from 0.25-1 μgmL⁻¹ (average 0.8 μgmL⁻¹), and the MICs with BromAc ranged from 0.004-0.25 μgmL⁻¹ (average 0.06 μgmL⁻¹) (Table I).

Duplicate assays confirmed this trend.

TABLE 1
MIC values for R. archizus and R. microsporus isolates treated with Amphotericin B,
Posaconazole and Isavuconazole alone and in combination with BromAc.

| | B | Amphoteracin B + | | Posaconazole + | | Isavuconazole + |
|---|---|---|---|---|---|---|
| | Amphoteracin | BromAC | Posaconazole | BromAC. | Isavuconazole | BromAC. |
| R. archizus 1 | 1 | 0.125 | 1 | 0.008 | 1 | 0.06 |
| R. archizus 2 | 0.5 | 0.004 | 1 | 0.008 | 1 | 0.125 |
| R. archizus 3 | 1 | 0.03 | 0.25 | 0.008 | 1 | 0.25 |
| R. arrhizus 4 | 0.5 | 0.016 | 0.25 | 0.03 | 1 | 0.25 |
| R. arrhizus 5 | 0.5 | 0.03 | 0.25 | 0.03 | 0.5 | 0.06 |
| R. arrhizus 6 | 1 | 0.03 | 0.5 | 0.08 | 1 | 0.25 |
| R. arrhizus 7 | 0.5 | 0.008 | 0.25 | 0.06 | 0.5 | 0.03 |
| R. archizus 8 | 0.5 | 0.03 | 0.25 | 0.03 | 1 | 0.03 |
| R. microsporus 1 | 1 | 0.125 | 1 | 0.25 | .2 | 0.5 |
| R. microsporus 2 | 1 | 0.125 | 1 | 0.125 | 0.5 | 0.125 |
| R. microsporus 3 | 0.25 | 0.008 | 0.25 | 0.016 | 0.5 | 0.06 |
| R. microsporus 4 | 0.5 | 0.06 | 0.25 | 0.06 | 1 | 0.25 |
| R. microsporus 5 | 0.5 | 0.03 | 0.25 | 0.016 | 0.5 | 0.125 |
| R. microsporus 6 | 0.5 | 0.016 | 0.25 | 0.004 | 0.5 | 0.06 |
| R. microsporus 7 | 0.5 | 0.016 | 0.25 | 0.03 | 0.5 | 0.125 |
| R. microsporus 8 | 0.5 | 0.016 | 0.25 | 0.016 | | 0.06 |

As can be seen from the results set out in Table 1, a reduction in MIC of 1-3 orders of magnitude is observed when BromAc is combined with the exemplified antifungal agents. This clearly demonstrates sensitization of the tested antifungal agents. As the concentration of BromAc were constant against the concentrations of antifungal agents, the inventors cannot conclude that a synergistic effect is occurring, however they can say that the MICs of the antifungal agents was reduced many fold.

### Example 4—Bromelain and Acetylcysteine in Combination (“BromAc”) Acts Synergistically with Amphotericin B, Posaconazole, Isavuconazole and Fluconazole to Inhibit Mucoromycete Growth

BromAc (5 mg/ml Acetylcysteine+12.5 μg/ml Bromelain) was used in combination with Amphotericin B, Isavuconazole, Posaconazole and Fluconazole. The MIC values of these four antifungal agents against the fungus described below were calculated and compared with their corresponding MIC values in the presence of BromAc, using the standard susceptibility assays described above.

The fungal strains set out in Table 2, all of which were sourced from a range of clinical isolates from Australian hospitals, were used in these experiments to gain a further understanding of the efficacy of BromAc in combination with Amphotericin B, Isavuconazole, Posaconazole and Fluconazole.

TABLE 2
Fungal strains tested

| | Number |
|---|---|
| Organism | of strains |
| Aspergillus | 7 |
| Candida auris | 5 |
| Cryptococcus | 10 |
| Candida glabrata | 5 |
| Candida kruseii | 6 |
| Candida tropicalis | 5 |
| Candida parapslosis | 1 |
| Cunninghamella | 2 |
| Fusarium | 4 |
| Licthemia | 3 |
| Lomentospora | 5 |
| Mucor circinelloides | 6 |
| Rhizopus arrhizus | 8 |
| Rhizopus microsporus | 11 |
| Scedosporium | 5 |

TABLE 3
Observed inhibitory effect (Strong = 5-10x
(or greater) inhibitory effect; Moderate = 1-5x
inhibitory effect; Possible = changes in the fungal
cells noted (morphological); and N/A = Not tested

| Organism | Amphotericin | Posaconazole | Isavuconazole | Fluconazole |
|---|---|---|---|---|
| Aspergillus | No effect | Moderate | Moderate | N/A |
| Candida aurus | Moderate | Strong | Strong | No effect |
| Cryptococcus | Strong | Strong | Strong | Strong |
| Candida glabrata | Strong | Strong | Strong | Strong |
| Candida kruseii | Strong | Strong | Strong | Strong |
| Candida tropicalis | Strong | Strong | Strong | Strong |
| Candida parapslosis | Strong | Strong | N/A | N/A |
| Cunninghamella | Possible | Moderate | Possible | N/A |
| Fusarium | No effect | No effect | No effect | N/A |
| Licthemia | Moderate | Moderate | Possible | N/A |
| Lomentospora | No effect | No effect | No effect | N/A |
| Mucor circinelloides | Strong | Strong | Strong | N/A |
| Rhizopus arrhizus | Strong | Strong | Strong | N/A |
| Rhizopus microsporus | Strong | Strong | Strong | N/A |
| Scedosporium | No effect | Possible | No effect | N/A |

The data from these experiments show:
Candida auris—more than 10× increase in 2 of 5 with Amphotericin, 4 of 5 Posaconazole and Isovuconazole
Cryptococcus—more than 10× with Amphotericin, Posaconazole, Isavuconazole and fluconazole in 5 of 5
Candida glabrata—more than a 10× increase in sensitivity in 5 of 5 isolates with amphotericin, in 5 of 5 with Posaconazole and 5 of 5 with Isavuconazole
Candida Krusei—more than 10× with amphotericin and Posaconazole
Candida tropicalis—more than a 10× increase in sensitivity in 4 of 5 with amphotericin and 4 of 5 with Posaconazole
Candida parapsilosis—almost 10× with amphotericin and more than 10× with Posaconazole
Cunninghamella—some synergy seen with Posaconazole
Mucor circinelloides—2 of 2 amphotericin, 1 of 2 Posaconazole and 2 of 2 Isavuconazole
Rhizopus Arrhizus—10× in 8 of 8 with amphotericin 7 of 8 with Posaconazole and 6 of 8 Isavuconazole
Rhizopus microspores—more than 10× in 9 of 10 with amphotericin and 9 of 10 with Posaconazole and Isavuconazole

### Example 5—Determination of Minimum Inhibitory Concentration (MIC) in Candida glabrata for BromAc Alone and in the Presence of Antifungals Using the Broth Dilution Susceptibility Test

These experiments were conducted to determine the effect of BromAc alone and in combination with existing antifungal drugs against Candida glabrata (clinical isolate).

The method used in these experiments is summarised below:

A. Preparation of inoculum:
1. Candida glabrata was grown on Sabouraud dextrose agar (SDA) at 37° C. for 24-48 hrs.
2. Following this, 5 colonies of 1 mm diameter were picked and suspended in 5 mL sterile saline (0.9% saline) and vortexed for 15 seconds.
3. Working suspension is prepared as a 1:100 dilution from stock suspension with RPMI 1640 culture medium.
4. The cell density is adjusted with a spectrophotometer to the transmittance produced by a 0.5 McFarland standard (0.08 to 0.1) OD at 625 nm wavelength.

### B. Preparation of Bromelain and N-Acetylcysteine Dilutions:

1. For Bromelain, a stock concentration of 1 mg/ml Bromelain solution was prepared in RPMI media and diluted to test concentrations of 62.4, 31.2, 15.6 and 7.8 μg/ml in RPMI 1640 media.
2. Acetylcysteine stock concentration is 200 mg/ml, diluted to 40, 20, 10, and 5 mg/ml concentrations in RPMI 1640 medium.
3. These concentrations are prepared at 2× of the required concentrations in the final assay plate.

### C. Preparation of Antifungal Stock Solutions:

### (Amphotericin B/Posaconazole/Fluconazole)

Antifungal stock concentrations are as follows—
1. Amphotericin B, Stock concentration: 250 μg/ml in water.
2. Posaconazole, Original concentration: 300 mg in 16.7 ml, Working stock concentration: 1 mg/ml in DMSO.
3. Fluconazole, Original concentration: 100 mg in 50 ml, Working stock concentration: 1 mg/ml in DMSO.
4. The final concentration was prepared in RPMI 1640 media and concentrations range was 0.01-0.5 μg/mL for amphotericin B, 0.01-2 μg/mL for posaconazole and 0.01-64 μg/mL for fluconazole.
5. These concentrations were prepared at 2× of the required concentrations in the final assay plate.

### D. Antifungal Assay (MIC) Using 96 Well Microtitration Plates.

1. Wells of the 96-well plates were inoculated with appropriate concentrations and volumes of drugs.
2. Sterility control and growth controls are included in each plate.
3. Each 96-well plate (except the control wells) contains 100 μL of double strength (twice the final antifungal drug) concentrations.
4. 100 μL of the fungal suspension is added to 96-well plates (except the sterility control wells).
5. The growth control wells contained 100 μL of sterile drug-free medium and 100 μL of inoculum suspension, while the sterility control wells contained 200 μl of sterile drug-free medium.
6. Plates are then incubated at 37° C. and read at 625,430, and 530 nm wavelengths.
7. Data from 24 hours and absorbance at 625 nm were used for analysis.

### Results

As shown in FIG. 2, a dose-dependent effect with Amphotericin B alone was observed against C. glabrata. A MIC of 1 ug/ml was found (MIC for amphotericin is measured at 90% growth inhibition).

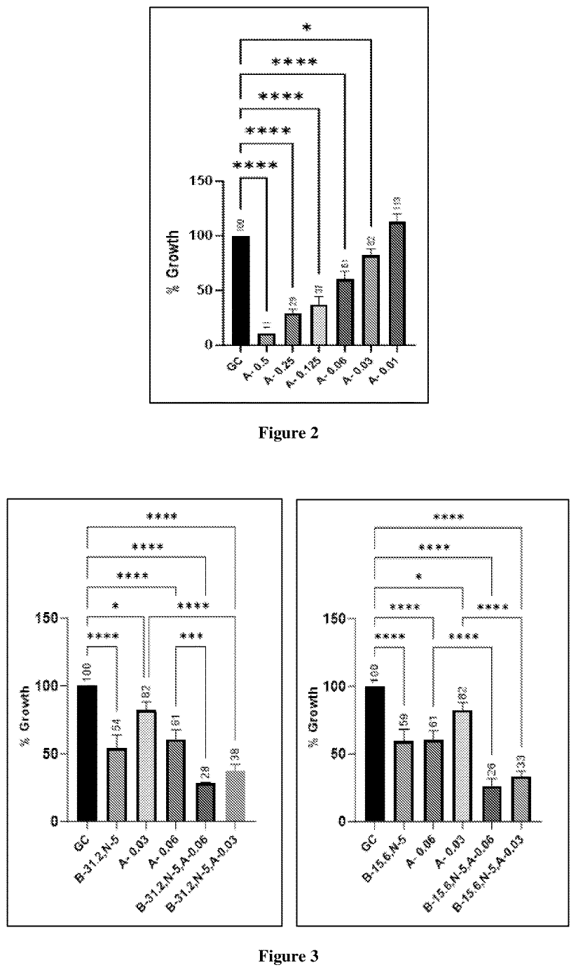

As shown in FIG. 3, BromAc on its own showed a decline or similar effects on the viability of C. glabrata compared to the Amphotericin B alone at the concentrations tested (FIGS. 3a and 3b, respectively). Adding Amphotericin B to BromAc significantly enhanced the effect on C. glabrata compared to the single treatments. Overall, all treatment groups were observed to inhibit fungal cell growth compared to growth controls.

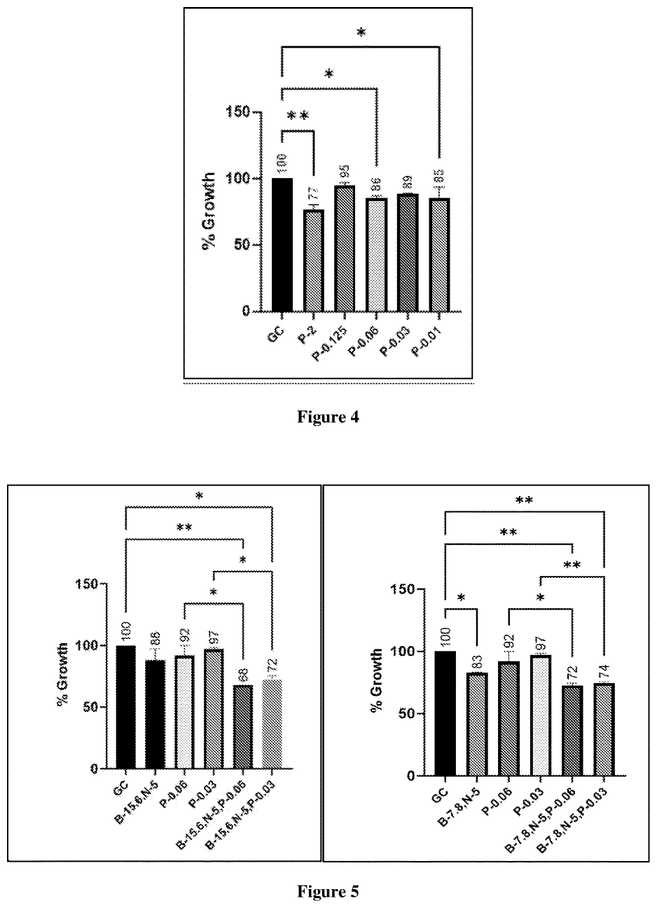

As shown in FIG. 4, compared to Amphotericin (i.e. FIG. 2), Posaconazole had a weaker antifungal effect on C. glabrata. Indeed, no dose response was observed across the concentration tested.

MIC for Posaconazole is 50% growth inhibition, which is determined to be 2 μg/ml; however, at this concentration, only a 25% decline in C. glabrata viability was observed over the growth control (FIG. 4). Lower concentration also showed a slight decline over GC and was further used to test the potency with the BromAc combination.

As can be seen in FIG. 5, BromAc showed a moderate decline in C. glabrata compared to Posaconazole alone at the concentrations tested (FIGS. 5a and 5b). The addition of Posaconazole to BromAc (higher and lower concentrations) showed significantly enhanced effect on C. glabrata (>25%) than Posaconazole alone.

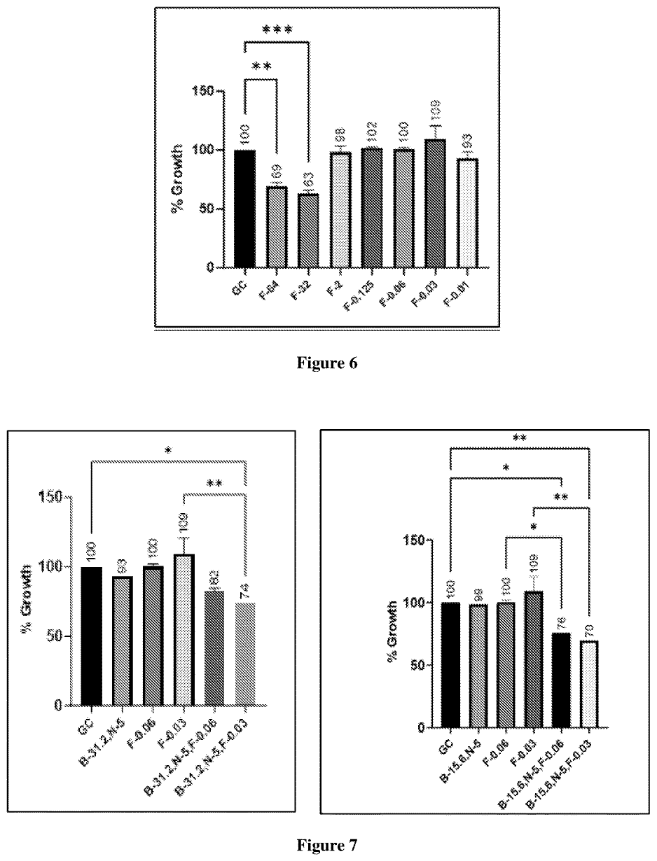

As shown in FIG. 6, like Posaconazole, the MIC for Fluconazole is determined to be 50% of growth inhibition of C. glabrata, observed at concentrations >32 μg/ml. Significant (30 to 40%) inhibition was found at 32 μg/ml and 64 μg/ml concentration with Fluconazole treatment compared to the growth control. A lower concentration of Fluconazole showed no change compared to the growth control (FIG. 6).

As can be seen in FIG. 7, BromAc showed no change in C. glabrata viability and was like Posaconazole alone at the doses tested (FIGS. 7a and 7b). However, like Posaconazole, the combination of Fluconazole and BromAc (higher and lower concentrations) showed significant activity on C. glabrata (>30%) than the Fluconazole treatment group (FIGS. 7a and 7b).

In conclusion, BromAc showed effective antifungal properties against C. glabrata on its own. Further, BromAc was found to enhance the effect of antifungals such as Amphotericin B, Posaconazole and Fluconazole, with concentrations hundred folds lower than the measured MIC values for C. glabrata.

### Example 6—Determination of Minimum Inhibitory Concentration (MIC) in Candida Krusei for BromAc Alone and in the Presence of Antifungals Using the Broth Dilution Susceptibility Test

These experiments were conducted to determine the effect of BromAc alone and in combination with existing antifungal drugs against Candida Krusei (ATCC strain 6258).

The method used in these experiments is essentially the same as that described above in Example 5.

### Results

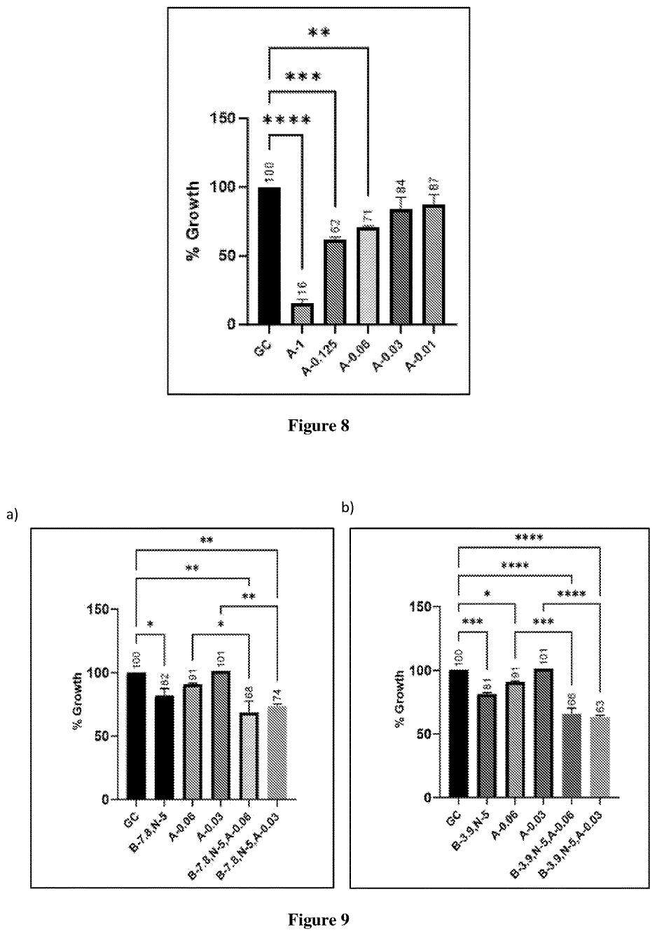

As shown in FIG. 8, a MIC close to 90% at 1 ug/ml was achieved with the treatment with Amphotericin B against C. krusei. A significant inhibition of the fungi was also observed at lower doses of Amphotericin B.

As shown in FIG. 9, BromAc on its own showed a decline in the viability of C. krusei compared to GC at both the higher (FIG. 9a) and lower (FIG. 9b) Bromelain concentrations tested. Adding Amphotericin B to BromAc significantly enhanced the effect on C. krusei (>25%) compared to the BromAc and Amphotericin B single treatments at the concentrations tested.

As shown in FIG. 10, Posaconazole showed significant (>50%) activity at its MIC of 1 ug/ml. Lower concentrations of Posaconazole 0.03 μg/ml showed no significant inhibition compared to GC (FIG. 10a).

BromAc on its own showed significant inhibition compared to GC. The addition of Posaconazole to BromAc did not significantly change the effect compared to BromAc alone; however, it was significant compared to the Posaconazole alone treatment at the concentration tested and also to GC (FIGS. 10a and 10b).

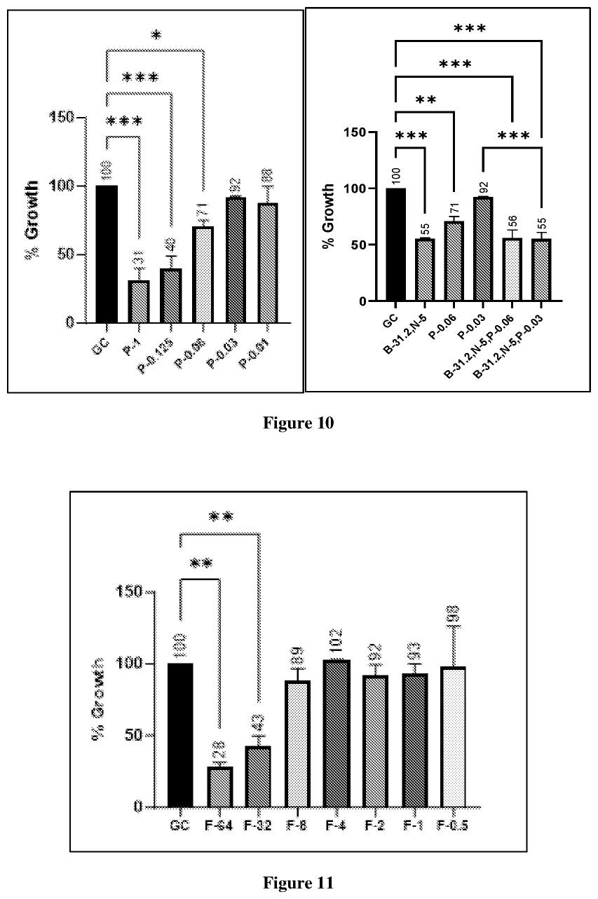

As shown in FIG. 11, the MIC for Fluconazole at 32 μg/ml achieved its 50% growth inhibition for C. krusei. A MIC of this calibre is classed as an ineffective agent. A lower concentration of Fluconazole showed no efficacy compared to the growth control.

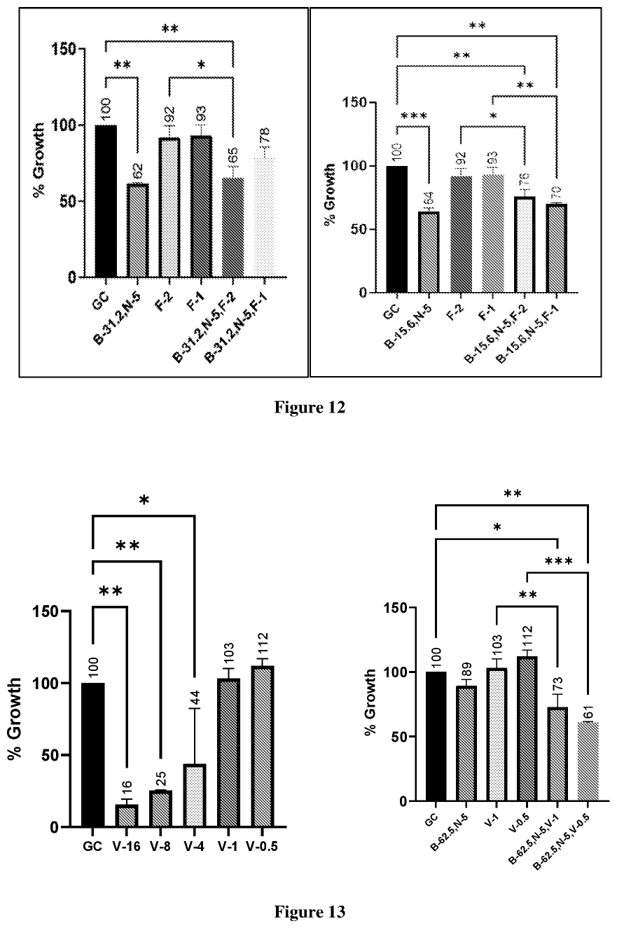

As shown in FIG. 12, BromAc on its own showed significant activity compared to GC. The addition of Fluconazole did not change the effect compared to BromAc alone treatment, however, the effect was significant compared to Fluconazole alone treatment (*p<0.05, **p<0.01) at the concentration tested and also to the GC (FIGS. 11a and b).

In conclusion, BromAc showed effective antifungal properties against C. krusei on its own. The combination of certain antifungal agents and BromAc also showed superior activity compared to their respective standalone concentrations.

### Example 7-Determination of Minimum Inhibitory Concentration (MIC) in Aspergillus fumigatus for BromAc Alone and in the Presence of Antifungals

These experiments were conducted to determine the efficacy of BromAc alone and in combination with existing antifungal drugs against Aspergillus fumigatus (ATCC strain 13697).

The method used in these experiments is essentially the same as that described above in Example 5. In these experiments, however, echinocandins such as Voriconazole and Caspofungin are used in place of the azole antifungals described in earlier examples.

### Results

As shown in FIG. 13a, a MIC of 2 μg/mL was observed with the treatment with Voriconazole against Aspergillus fumigatus. At lower concentrations, however (e.g. 1 μg/ml and 0.5 μg/ml), almost no inhibitory effect was observed (FIG. 13a).

As can be seen in FIG. 13b, BromAc on its own showed a modest decline in the viability of Aspergillus fumigatus compared to GC. However, the combination of BromAc and Voriconazole (even at low doses of Voriconazole) significantly enhanced the effect on Aspergillus fumigatus (p<0.01) compared to the BromAc and Voriconazole single treatments at the concentrations tested.

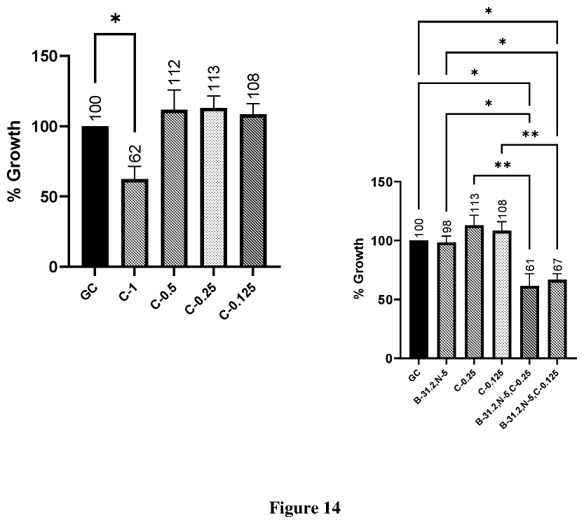

Referring now to FIGS. 14a and 14b, a similar effect can be seen for Caspofungin. As can be seen in FIG. 13a, a MIC of >1 μg/ml was observed with the treatment with Caspofungin against Aspergillus fumigatus. At lower concentrations, however (e.g. 0.25 μg/ml and 0.125 μg/ml), almost no inhibitory effect was observed.

As can be seen in FIG. 14b, BromAc on its own showed a modest decline in the viability of Aspergillus fumigatus compared to GC. However, the combination of BromAc and Caspofungin (even at low doses of Caspofungin) significantly enhanced the effect on Aspergillus fumigatus (p<0.01) compared to the BromAc and Caspofungin single treatments at the concentrations tested.

These data indicate that the present invention has applicability to other classes of antifungal agents, including echinocandins, and clearly demonstrate that, when added in combination with BromAc, lower concentrations of antifungal can be used. Administering lower concentrations of drugs (having known side effects) is important for a systemic approach). The limitations to antifungal include the balance between efficacy and toxicity as seen specifically with voriconazole.

The data is not shown, but the inventors have observed similar effects using these antifungal drugs (as well as Micafungin) against Candida auris and Aspergillus flavus.

The inventors believe that these data, showing strong inhibitory effects of combinations in accordance with embodiments of the present invention in a number of fungal types, lead to a reasonable prediction of the therapeutic indications described herein. Further experiments, some of which are already underway, will confirm the inventors' expectations.

As described herein, the present invention provides compositions and methods for treating fungal infections/diseases, in which methods a glycoprotein affecting protease, such as bromelain, is administered. Embodiments of the present invention provide a number of advantages over existing therapies, some of which are described above.

It will be understood to persons skilled in the art of the invention that many modifications may be made without departing from the spirit and scope of the invention. All such modifications are intended to fall within the scope of the following claims.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1. A method for treating a fungal infection in a patient, the method comprising administering to the patient a therapeutically effective combination of a glycoprotein affecting protease and an antifungal agent.

2. The method of claim 1, wherein the fungal infection is caused by a fungus from a fungal genus selected from one or more of the group consisting of: Rhizopus, Mucor, Rhizomucor, Cryptococcus, Candida, Syncephalastrum, Cunninghamella, Apophysomyces, Aspergillus, Histoplasma, Pneumocystis and Lichtheimia.

3. The method of claim 1 or claim 2, wherein the fungal infection is caused by a fungus selected from one or more of the group consisting of: Rhizopus arrhizus, Rhizopus microsporus, Aspergillus fumigatis, Aspergillus flavus, Aspergillus niger, Fusarium solani, Candida krusei, Candida auris, Candida parapslosis, Candida tropicalis, Candida glabrata, Candida albicans, Cryptococcus gattii, Cryptococcus neoformans, Cunninghamella bertholletiae, Lichtheimia corymbifera, Mucor circinelloides, Histoplasma capsulatum, Pneumocystis jirovecii and Microsporidia.

4. The method of any one of claims 1 to 3, wherein the fungal infection is selected from one or more of the group consisting of: mucormycosis (cutaneous mucormycosis, Rhino-cerebral mucormycosis and pulmonary mucormycosis), histoplasmosis, cryptococcosis, pneumocystis pneumonia, candidiasis and aspergillosis.

5. The method of any one of claims 1 to 4, wherein the glycoprotein affecting protease and the antifungal agent are administered to the patient simultaneously or sequentially.

6. The method of any one of claims 1 to 4, wherein the glycoprotein affecting protease and the antifungal agent are co-administered to the patient.

7. The method of any one of claims 1 to 6, wherein the glycoprotein affecting protease and the antifungal agent are administered to the patient via different routes of administration.

8. The method of any one of claims 1 to 7, wherein the glycoprotein affecting protease and the antifungal agent are administered to the patient locally, systemically, intravenously, by inhalation, by nebulization, by intratracheal injection, by intracavitary injection, by intraperitoneal injection, by intrapleural injection, by intraocular injection, by injection into the parenchyma or by injection into the cerebrospinal fluid via the intracerebroventricular or intrathecal (cisternal or lumbar) route.

9. The method of any one of claims 1 to 8, wherein the glycoprotein affecting protease is a cysteine protease.

10. The method of any one of claims 1 to 9, wherein the glycoprotein affecting protease is selected from one or more of the group consisting of: bromelain, papain, ficain, actinidain, zingibain, fastuosain and ananain.

11. The method of any one of claims 1 to 10, wherein the antifungal agent is selected from one or more of the group consisting of: amphotericin B, nystatin, caspofungin, micofungin, isavuconazole, posaconazole, ketaconazole, itraconazole, voriconazole, fluconazole and terbinafine.

12. The method of any one of claims 1 to 11, further comprising administering to the patient a disulphide bond breaking agent in combination with the glycoprotein affecting protease and the antifungal agent.

13. The method of claim 12, wherein the disulphide bond breaking agent is selected from one or more of the group consisting of: N-acetylcysteine, cysteamine, carbocysteine, bucillamine, dithiobutylamine (DTBA) and glutathione.

14. The method of claim 12 or claim 13, wherein the glycoprotein affecting protease and disulphide bond breaking agent are administered simultaneously or sequentially.

15. The method of any one of claims 1 to 14, further comprising administering to the patient one or more additional agents that degrade fungal cell walls or cellular DNA components.

16. The method of claim 15, wherein the one or more additional therapeutic agent is a DNase.

17. The method of any one of claims 1 to 16, further comprising administering to the patient one or more additional therapeutic agents.

18. The method of claim 17, wherein the one or more additional therapeutic agents are selected from the group consisting of antiviral agents, antibacterial agents, bronchodilators and expectorants.

19. A method for sensitizing a fungus which is the cause of a fungal infection in a patient to an antifungal agent, wherein the fungus has a glycoprotein-containing fungal cell wall, the method comprising administering to the patient a glycoprotein affecting protease.

20. A method for sensitizing a fungus having a glycoprotein-containing fungal cell wall to an antifungal agent,
the method comprising contacting the fungus with a glycoprotein affecting protease.

21. A method, for treating a fungal infection in a patient, the method comprising administering to the patient a therapeutically effective combination of a glycoprotein affecting protease and a disulphide bond breaking agent.

22. The use of a glycoprotein affecting protease to sensitize a fungus having a glycoprotein-containing fungal cell wall, whereby efficacy of an antifungal agent is increased.

23. A composition comprising a glycoprotein affecting protease and an antifungal agent.

24. A synergistic combination of a glycoprotein affecting protease and an antifungal agent.

25. A synergistic combination of a glycoprotein affecting protease and an antifungal agent, when used to sensitize a fungus having a glycoprotein-containing fungal cell wall to an antifungal agent.

26. The use of a combination of a glycoprotein affecting protease and an antifungal agent for the preparation of a medicament for the treatment of a fungal infection in a patient.

27. A combination of a glycoprotein affecting protease and an antifungal agent for use in medicine.

28. A combination of a glycoprotein affecting protease and an antifungal agent for use in the treatment of a fungal infection in a patient.
